(19) United States
(12) Patent Application Publication
(10) Pub. No.: US 2024/0320842 A1
(43) Pub. Date: Sep. 26, 2024

(54) PROVIDING A RESULT DATASET

(71) Applicant: Siemens Healthineers AG, Forchheim (DE)

(72) Inventors: Michael Manhart, Fürth (DE); Stanislav Tashenov, Heroldsbach (DE)

(21) Appl. No.: 18/610,895

(22) Filed: Mar. 20, 2024

(30) Foreign Application Priority Data
Mar. 20, 2023 (DE) 10 2023 202 451.9

(51) Int. Cl. G06T 7/33; G06T 11/60; G06V 10/74

(52) U.S. Cl. CPC G06T 7/337; G06T 11/60; G06V 10/761; G06T 2207/30101; G06T 2210/41; G06V 2201/031

(57) ABSTRACT

A method for providing a result dataset includes capturing a vessel dataset. The vessel dataset has time-resolved images of at least one vessel section of an examination object in a number of physiological phases. The method includes capturing an object dataset. The object dataset has an image of a medical object in the examination object in at least one physiological phase of the number of physiological phases. The method includes identifying a corresponding image in the vessel dataset in each case for the images of the object dataset with matching physiological phase, and providing the result dataset by at least partial overlaying and/or mixing of the images of the object dataset with the corresponding images of the vessel dataset.

This application claims the benefit of German Patent Application No. DE 10 2023 202 451.9, filed on Mar. 20, 2023, which is hereby incorporated by reference in its entirety.

### BACKGROUND

The present embodiments relate to a method for providing a result dataset, to a provision unit, to a medical imaging device, and to a computer program product.

In interventional radiology, a navigation with guidewires and/or microcatheters and/or a placing of vessel implants (e.g., a stent) is frequently carried out using fluoroscopy. Vessel sections that are not contrasted are not visible under fluoroscopy. For detection of vessel sections, a digital subtraction angiography (DSA) may be applied, where at least two x-ray images recorded in a time sequence that each depict an image of a common examination region including the vessel section are subtracted from one another. Further, in a DSA, a distinction is frequently made between a mask phase for recording at least one mask image and a fill phase for recording at least one fill image. In this case, the mask image may depict the examination region uncontrasted (e.g., without contrast). The fill image may further depict the examination region contrasted (e.g., while contrast is arranged in the examination region). As a result of the DSA, a difference image is frequently provided by subtraction of mask and fill image. This often enables the components in the difference image that are irrelevant and/or disruptive for a treatment and/or diagnosis that, for example, are unchanged over time to be reduced and/or removed.

The difference images may be overlaid with a fluoroscopy image, with the fluoroscopy image depicting an image of a medical object (e.g., the guidewire and/or microcatheter and/or the implant) arranged in the examination region (e.g., in the vessel section).

To reduce image artifacts (e.g., movement artifacts), the recording of the DSA may be made during a breath-hold. Disadvantageously, a movement of inner organs of the examination object (e.g., a liver, kidneys, and/or lungs) during the fluoroscopy may lead to a low match with the difference image. To reduce these types of discrepancies, roadmapping methods may be applied, which register the DSA with the fluoroscopy. With bone structures, the registration may frequently achieve a good result, but the registration often fails, however, with soft tissue that is moving relative to the bone structures.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved imaging-based supervision of a medical object arranged in an examination object is provided.

Independent of the grammatical term usage, individuals with male, female or other gender identities are included within the term.

The present embodiments relate, in a first aspect, to a method for providing a result dataset. In this aspect, a vessel dataset is captured. The vessel dataset has time-resolved images of at least one vessel section of an examination object in a number of physiological phases. An object dataset that has an image of a medical object in the examination object in at least one matching physiological phase of the number of physiological phases is further captured. Further, a corresponding image in the vessel dataset for the images of the object dataset with matching physiological phase is identified in each case. Further, the result dataset may be provided by at least part overlaying and/or mixing of the images of the object dataset with the corresponding images of the vessel dataset.

The method acts described here may, at least in part, be carried out simultaneously or one after the other. Further, the method acts described here may be computer-implemented, at least in part (e.g., completely).

The examination object may, for example, be a human and/or animal male or female patient and/or may be an examination phantom.

The capture of the vessel dataset may include a receipt and/or recording of the vessel dataset. The receipt of the vessel dataset may, for example, include a capture and/or redout of a computer-readable data memory and/or a receipt from a data memory unit (e.g., a database). The vessel dataset may further be provided by a provision unit of a medical imaging device. As an alternative or in addition, the vessel dataset may be recorded by the medical imaging device.

The medical imaging device for recording of the vessel dataset may include a medical x-ray device (e.g., a medical C-arm x-ray device and/or cone-beam computed tomography system (cone-beam CT, CBCT)) and/or a computed tomography system (CT system) and/or a magnetic resonance tomography system (MRT system) and/or a Positron Emission Tomography system (PET system) and/or an ultrasound device.

The vessel dataset may include a number of two-dimensional (2D) and/or three-dimensional (3D) spatially-resolved images of the at least one vessel section, which depict the at least one vessel section in a time-resolved manner. The at least one vessel section may, for example, include an artery and/or vein and/or a vessel malformation (e.g., an aneurysm). The vessel dataset may depict the at least one vessel section in a time-resolved manner in the number of physiological phases. The number of physiological phases may include temporally (e.g., periodically) repeating phases of a physiological movement (e.g., an organ movement and/or breath movement and/or heart movement) of the examination object. In one embodiment, the number of physiological phases may be consecutive and/or show an image of the period of the physiological movement at least in part (e.g., completely). For example, the vessel dataset may be recorded during free breathing of the examination object. In this case, the vessel dataset may depict the at least one vessel section within at least one breathing cycle in a time-resolved manner.

The capture of the object dataset may include a receipt and/or recording of the object dataset. The receipt of the object dataset may, for example, include a capture and/or readout of a computer-readable data memory and/or a receipt from a data memory unit (e.g., a database). The object dataset may further be provided by a provision unit of a medical imaging device. As an alternative or in addition, the object dataset may be recorded by the medical imaging device. The vessel dataset and the object dataset may be recorded by the same or different medical imaging devices.

The object dataset may include at least one (e.g., a number of) two-dimensional (2D) and/or three-dimensional (3D) spatially-resolved images of the medical object that is arranged in the examination object (e.g., in the at least one vessel section). The medical object may, for example, include a surgical and/or diagnostic instrument (e.g., a catheter and/or guidewire and/or an endoscope) and/or an implant (e.g., a stent).

The medical object may be introduced via a body opening and/or an insertion port at an entry point into the examination object (e.g., the examination region and/or a hollow organ). In this case, at least one section of the medical object (e.g., a distal section) is arranged in the examination object.

In one embodiment, the object dataset may depict an image of the medical object arranged in the examination object in at least one (e.g., in a number of or in each of the number of physiological phases). For example, the object dataset may depict the medical object arranged in the examination object in at least one matching physiological phase of the number of physiological phases that are imaged in the vessel dataset. The object dataset may be recorded before or after the vessel dataset in time.

In one embodiment, a corresponding image may be identified in the vessel dataset for each of the images of the object dataset in each case. The correspondence may relate in this case to a matching physiological phase of the number of physiological phases in each case. The identification of the corresponding image in each case may be undertaken (e.g., automatically) with the aid of recording parameters of the vessel dataset and of the object dataset (e.g., recording times) and/or with the aid of a physiological movement signal and/or with the aid of geometrical and/or anatomical features that are depicted in the vessel dataset and the object dataset. If the object dataset depicts the medical object in the examination object in a number of physiological phases, then a corresponding image in the vessel dataset with matching physiological phase may be identified for each of the images of the medical object in the object dataset in each case.

The matching physiological phases may refer to physiologically equivalent phases at various points in time (e.g., recording times, such as a breathing-in phase or breathing-out phase of a breath movement and/or a systolic or diastolic phase of a heart movement).

The vessel dataset and/or the object dataset may each include a number of image points (e.g., pixels or voxels) with image values (e.g., attenuation values and/or intensity values) that depict the examination object.

In one embodiment, the result dataset may be provided by at least partial (e.g., regional and/or semitransparent and/or color-coded) overlaying and/or mixing of the images of the object dataset, with the respective corresponding images of the vessel dataset. The provision of the result dataset may include a storage on a computer-readable memory medium and/or a display on a display unit and/or a transmission to a provision unit. For example, a graphical representation of the result dataset may be displayed by the display unit.

The method may make possible an improved imaging-based supervision of the medical object arranged in the examination object.

In a further form of embodiment of the method, the object dataset may have time-resolved images of the medical object in the examination object in a number of physiological phases. A corresponding image in the vessel dataset may further be identified in each case for each of the images of the object dataset with matching physiological phase.

In one embodiment, the object dataset may include images of the medical object for a subset or for each of the number of physiological phases depicted in the vessel dataset. A corresponding image in the vessel dataset may further be identified in each case for each of the images of the object dataset with matching physiological phase. The result dataset may be provided by at least partial overlaying and/or mixing in each case of an image of the object dataset with the corresponding image of the vessel dataset.

The embodiment may make possible a supervision of the medical object in the examination object during the number of physiological phases with few movement artefacts (e.g., with minimized movement artefacts).

In a further form of embodiment of the method, the capture of the vessel dataset may include a recording of a first mask dataset in a first mask phase and of a fill dataset in a fill phase. In this case, the first mask dataset and the fill dataset each have time-resolved images of a common examination region in matching physiological phases. The common examination region may further include the at least one vessel section. Further, in the fill phase, a contrast may be arranged in the at least one vessel section and imaged in the fill dataset. The vessel dataset may further be provided as the difference between images of the fill dataset and of the first mask dataset with matching physiological phase in each case.

In one embodiment, the first mask dataset and the fill dataset may be recorded by the same medical imaging device. In this case, the first mask dataset may be recorded in the first mask phase (e.g., in a first period of time). In this case, in the first mask phase (e.g., in the first period of time), essentially no contrast is arranged in the at least one vessel section. The fill dataset may further be recorded in the fill phase (e.g., in a further period of time before or after the first period of time). In this case, in the fill phase (e.g., in the further period of time), the contrast (e.g., an x-ray-opaque contrast) may be arranged in the at least one vessel section. The common examination region may include a spatial section of the examination object (e.g., a volume that includes the at least one vessel section). Further, the first mask dataset and the fill dataset may depict the common examination region in a time-resolved manner and in each of the number of physiological phases. In one embodiment, the images of the fill dataset may depict the at least one contrasted vessel section (e.g., the contrast means arranged in the at least one vessel section) in the matching physiological phases. The images of the first mask dataset may further show images the essentially uncontrasted examination region in the matching physiological phases.

The vessel dataset may be provided as the (e.g., image point by image point) difference between the images of the fill dataset and of the first mask dataset with matching physiological phase in each case. For example, the vessel dataset may in each case have a vessel difference image for each pair of images of the fill dataset and of the first mask dataset with matching physiological phase.

In one embodiment, a vessel difference image may be identified in the vessel dataset for each of the images of the object dataset with matching physiological phase. The result dataset may further be provided by at least partial overlaying and/or mixing of the images of the object dataset with the corresponding vessel difference images of the vessel dataset.

The form of embodiment may make possible an improved supervision of the medical object in relation to the at least one vessel section.

In a further form of embodiment of the method, the capture of the object dataset may include a recording of an initial object dataset in an object phase. In this case, the initial object dataset may have an image of the common examination region of the examination object in the at least one physiological phase. Further, the medical object may be arranged in the object phase in the common examination region and be depicted in the initial object dataset. The object dataset may further be provided as the difference between images of the initial object dataset and of the first mask dataset with matching physiological phase in each case.

The initial object dataset may be recorded in the object phase (e.g., in a period of time different from the mask and/or fill phase). In this case, in the object phase, the medical object may be arranged in the common examination region (e.g., in the at least one vessel section). The initial object dataset may depict the common examination region (e.g., the medical object) in a time-resolved manner within the object phase and in the at least one matching physiological phase. For example, the initial object dataset may include one or a number of images of the common examination region with the medical object arranged therein within the object phase.

The object dataset may be provided as the (e.g., image point by image point) difference between the at least one image of the initial object dataset and of the first mask dataset with matching physiological phase in each case (e.g., in an efficient manner as regards time and/or x-ray dose). For example, the vessel dataset may in each case have an object difference image for each pair of images of the initial object dataset and of the first mask dataset with matching physiological phase.

In one embodiment, a vessel difference image in the vessel dataset may be identified in each case for the object difference images of the object dataset with matching physiological phase. The result dataset may further be provided by at least partial overlaying and/or mixing of the object difference images of the object dataset with the corresponding vessel difference images of the vessel dataset.

The form of embodiment may make possible a removal from the initial object dataset and the fill dataset of structures of the examination object that do not change over time. This enables an improved supervision of the medical object in relation to the at least one vessel section to be made possible.

In a further form of embodiment of the method, the capture of the object dataset may include a recording of a second mask dataset in a second mask phase and of an initial object dataset in an object phase. In this case, the second mask dataset and the initial object dataset may each have an image of a common examination region of the examination object in at least one matching physiological phase. In this case, the medical object may be arranged in the object phase in the common examination region and be imaged in the initial object dataset. The object dataset may further be provided as the difference between images of the initial object dataset and the second mask dataset with matching physiological phase in each case.

In one embodiment, the second mask dataset and the initial object dataset may be recorded by the same medical imaging device. In this case, the second mask dataset may be recorded in the second mask phase (e.g., in a first period of time). In this case, in the second mask phase (e.g., in the first period of time), essentially no contrast is arranged in the at least one vessel section. The initial object dataset may be recorded in the object phase (e.g., in a period of time different from the second mask phase). In this case, in the object phase, the medical object may be arranged in the common examination region (e.g., in the at least one vessel section). The initial object dataset may image the common examination region (e.g., the medical object) within the object phase in a time-resolved manner and in the at least one matching physiological phase. For example, the initial object dataset may include one or a number of images of the common examination region (e.g., of the at least one vessel section), with the medical object arranged therein, within the object phase.

The object dataset may be provided (e.g., image point by image point) as the difference between the at least one image of the initial object dataset and of the second mask dataset with matching physiological phase in each case. For example, the object dataset may in each case have an object difference image for each pair of images of the initial object dataset and of the second mask dataset with matching physiological phase.

In one embodiment, one image in each case (e.g., a vessel difference image) may be identified in the vessel dataset for the object difference images of the object dataset with matching physiological phase. The result dataset may further be provided by at least partial overlaying and/or mixing of the object difference images of the object dataset with the corresponding images of the vessel dataset.

The form of embodiment may make possible a removal from the initial object dataset of structures of the examination object that do not change over time. This enables an improved supervision of the medical object in relation to the at least one vessel section to be made possible.

In a further form of embodiment of the method, the images to be subtracted in each case with the respective matching physiological phase may be identified with the aid of a similarity metric that assesses a similarity of images of various datasets.

In one embodiment, the similarity metric may be robust in relation to changes of capture parameters of the imaging device for capturing the vessel dataset and of the object dataset (e.g., tube parameters of a medical x-ray device). The similarity metric may assess the similarity of the images of various datasets (e.g., of the vessel dataset and of the object dataset). For example, the similarity metric for each pair of images of the various datasets may provide a similarity value in each case, which assesses the similarity of the pairs of images. The similarity metric may, for example, include a zero-normalized cross-correlation (NCC). In this case, pairs of images of the various datasets in each case, which have a maximum similarity value, may be identified as the respective images to be subtracted with the respective matching physiological phase. As an alternative or in addition, the similarity metric may be based on a total intensity variation of a subtraction result of the images of various datasets.

This enables a robust identification of the respective images of the various datasets to be subtracted to be made possible.

In a further form of embodiment of the method, the respective images to be subtracted may be registered with one another.

The images to be subtracted may include the images of the first mask dataset, of the second mask dataset, of the fill dataset, of the initial object dataset, of the vessel dataset and/or of the object dataset. The respective images to be subtracted may further include the vessel difference images and/or the object difference images.

In one embodiment, the respective images to be subtracted are registered with one another (e.g., based on common geometrical and/or anatomical features). The common geometrical features may, for example, include edges and/or contours and/or a marker structure and/or a contrast transition that is depicted in the images to be subtracted. The common anatomical features may, for example, include a tissue boundary and/or an anatomical landmark (e.g., an ostium) and/or an implant that is depicted in the images to be subtracted.

The registering of the images to be subtracted may include an application of a, for example, rigid or non-rigid, transformation (e.g., a translation and/or rotation and/or scaling and/or deformation) to one or to both of the images to be subtracted. A deviation between the common geometrical and/or anatomical features is reduced (e.g., minimized).

This enables movement artifacts to be reduced in the result dataset.

In a further form of embodiment, the capture of the vessel dataset may include a recording of a fill dataset in a fill phase. In this case, the capture of the object dataset may include a recording of an initial object dataset in an object phase. The fill dataset and the initial object dataset may each have an image of a common examination region of the examination object in at least one matching physiological phase. The common examination region may further include at least one vessel section. Further, in the fill phase, a contrast may be arranged in the at least one vessel section and depicted in the fill dataset. Further, the medical object may be arranged in the object phase in the common examination region and be depicted in the initial object dataset. In this case, the result dataset may be provided by at least partial overlaying and/or mixing of the images of the initial object dataset with the corresponding images of the fill dataset.

The initial object dataset and the fill dataset may, for example, have all features and characteristics that have already been described in relation to alternate forms of embodiment and vice versa.

In one embodiment, the result dataset may be provided by at least partial (e.g., region-by-region and/or semitransparent and/or color-coded) overlaying and/or mixing of the images of the initial object dataset of the object dataset with the respective corresponding images of the fill dataset.

The form of embodiment may make possible a removal from the initial object dataset of structures of the examination object and of the fill dataset that do not change over time. This enables an improved supervision of the medical object in relation to the at least one vessel section to be made possible.

In a further form of embodiment of the method, the physiological phases may include various phases of a breath movement and/or heart movement of the examination object.

For example, the physiological phases may include a breathing-in phase and a breathing-out phase of the examination object. As an alternative or in addition, the physiological phases may include various phases of the heart movement of the examination object (e.g., a diastolic phase and a systolic phase).

A half breath cycle between a complete breathing-in and breathing-out phase may be used as the minimal mask phase. The fill phase may include a subsequent breath phase (e.g., a subsequent breath cycle).

This enables movement artifacts in the provision of the result dataset that are brought about by the breath movement and/or heart movement of the examination object to be reduced (e.g., minimized).

In a further form of embodiment of the method, the medical object may include a diagnostic and/or surgical instrument and/or an implant.

The diagnostic and/or surgical instrument may, for example, include a catheter and/or guidewire and/or an endoscope that has been arranged in the examination object before the beginning of the method. As an alternative, the medical object may include an implant (e.g., a stent) that has been arranged in the examination object before the beginning of the method.

The form of embodiment may make possible an improved supervision of the current positioning of the diagnostic and/or surgical instrument and/or of the implant in relation to the at least one vessel section.

In a further form of embodiment of the method, the identification of respective one corresponding image in the vessel dataset for the images of the object dataset with matching physiological phase may be based on a further similarity metric that assesses a similarity between images of the vessel dataset and images of the object dataset.

The further similarity metric may be the same as or different from the similarity metric for identification of the respective images to be subtracted. In one embodiment, the further similarity metric may be robust in relation to changes of capture parameters of the imaging device for capture of the vessel dataset and of the object dataset (e.g., tube parameters of a medical x-ray device). The further similarity metric may assess the similarity between images of the vessel dataset and images of the object dataset. For example, the further similarity metric may provide a similarity value for each pair of images of the vessel dataset and images of the object dataset in each case, which depicts the similarity of the imaging pairs. The further similarity metric may, for example, include a zero-normalized cross-correlation (NCC). In this case, pairs of images of the vessel dataset and of the object dataset that exhibit a maximum similarity value may be identified as the respective images to be subtracted with the respective matching physiological phase. As an alternative or in addition, the further similarity metric may be based on a total intensity variation.

The form of embodiment may make possible an improved (e.g., robust) identification of the respective one image in the vessel dataset corresponding to the images of the object dataset with matching physiological phase.

In a further form of embodiment of the method, the provision of the result dataset may include a registration of the images of the object dataset with the corresponding images of the vessel dataset.

In one embodiment, the images of the object dataset may be registered with the respective corresponding images of the vessel dataset (e.g., based on common geometrical and/or anatomical features). The common geometrical features may, for example, include edges and/or contours and/or a marker structure and/or a contrast transition that is depicted in the images of the object dataset and in the respective corresponding images of the vessel dataset. The common anatomical features may, for example, include a tissue boundary and/or an anatomical landmark (e.g., an ostium) and/or an implant that is depicted in the images of the object dataset and thus in the respective corresponding images of the vessel dataset.

The registering of the images of the object dataset with the respective corresponding images of the vessel dataset may include an application of a, for example, rigid or non-rigid transformation (e.g., a translation and/or rotation and/or scaling and/or deformation) to one or both of the images of the object dataset and the thus respective corresponding images of the vessel dataset. A deviation between the common geometrical and/or anatomical features is reduced (e.g., minimized).

This enables misalignment artifacts (e.g., movement artifacts) in the result dataset to be minimized.

In a further form of embodiment of the method, a physiological movement signal, featuring information about a current physiological phase of the examination object, may be received. In this case, the vessel dataset and/or the object dataset may be captured depending on the physiological movement signal.

The receipt of the movement signal may, for example, include an acquisition and/or reading out of a computer-readable data memory and/or a receipt from a data memory unit (e.g., a database). The movement signal may further be provided by a provision unit of a physiological sensor (e.g., an electrocardiograph (EKG) and/or a breath sensor and/or a pulse sensor and/or a movement sensor) and/or a sensor for detecting a positioning of the examination object (e.g., an electromagnetic and/or optical and/or acoustic and/or mechanical sensor). The movement signal may feature information about the current physiological phase of the examination object.

In one embodiment, the vessel dataset and/or the object dataset may be captured depending on the physiological movement signal (e.g., triggered depending on the current physiological phase of the examination object).

This enables it to be provided that the object dataset has at least one image of the medical object in the examination object in the at least one matching physiological phase.

The present embodiments relate in a second aspect to a provision unit that is configured to carry out a method of the present embodiments.

In this case, the provision unit may include a computing unit, a memory unit, and/or an interface. The provision unit may be configured to carry out a method for providing a result dataset, in which the interface, the computing unit, and/or the memory unit are configured to carry out the corresponding method acts.

For example, the interface may be configured for capture of the vessel dataset and/or of the object dataset and/or for provision of the result dataset. The computing unit and/or the memory unit may further be configured for identification of the respective one corresponding image in the vessel dataset for the images of the object dataset with matching physiological phase.

The advantages of the proposed provision unit essentially correspond to the advantages of the proposed method for providing a result dataset. Features, advantages, or alternate forms of embodiment mentioned here may likewise be transferred to the other claimed subject matter and vice versa.

The present embodiments relate, in a third aspect, to a medical imaging device including a proposed provision unit. In this case, the imaging device is configured for capture of the vessel dataset and of the object dataset.

The advantages of the imaging device of the present embodiments essentially correspond to the advantages of the method for providing a result dataset of the present embodiments. Features, advantages, or alternate forms of embodiment mentioned here may likewise be transferred to the other subject matter and vice versa.

The medical imaging device for capture (e.g., recording) of the vessel dataset and of the object dataset may include a medical x-ray device (e.g., a medical C-arm x-ray device and/or a cone-beam computed tomography system (cone-beam CT, CBCT)) and/or a computed tomography system (CT system) and/or a magnetic resonance tomography system (MRT system) and/or a Positron Emission Tomography system (PET system) and/or an ultrasound device.

The present embodiments relate, in a fourth aspect, to a computer program product with a computer program that is able to be loaded directly into a memory of a provision unit, with program sections for carrying out all acts of a method for providing a result dataset of the present embodiments when the program sections are executed by the provision unit. The computer program product may in this case be software with a source code that still has to be compiled and linked or that only has to be interpreted or may include executable software code that only has to be loaded into the provision unit to be executed. The computer program product enables the method for providing a result dataset using a provision unit to be carried out quickly, identically repeatably, and robustly. The computer program product is configured so that the computer program product may execute the method acts of the present embodiments by the provision unit.

The computer program product is, for example, stored on a computer-readable memory medium (e.g., a non-transitory computer-readable storage medium) or is held on a network or server, from where the computer program product may be loaded into the processor of a provision unit that may be configured linked directly to the provision unit or as part of the provision unit. Further, control information of the computer program product may be stored on an electronically-readable data medium. The control information of the electronically-readable data medium may be configured such that when the data medium is used in a provision unit, the control information carries out a method of the present embodiments. Examples of electronically-readable data media are a DVD, a magnetic tape, or a USB stick, on which electronically-readable control information (e.g., software) is stored. When this control information is read from the data medium and stored in a provision unit, all forms of embodiment of the method previously described may be carried out.

A largely software-based realization has the advantage that even provision units previously used may be upgraded in a simple manner by a software update in order to work according to the present embodiments. Such a computer program product, as well as the computer program, may, if necessary, include additional elements such as, for example, documentation and/or additional components, as well as hardware components, such as, for example, hardware keys (e.g., dongles etc.) for use of the software.

### BRIEF DESCRIPTION OF THE DRAWINGS

Embodiments of the invention are shown in the drawings and will be explained in greater detail below. The same reference characters are used for the same features in different figures. In the figures:

FIGS. 1 to 6 show schematic diagrams of various forms of embodiment of a method for provision of a result dataset;

FIG. 7 shows a schematic diagram of a proposed provision unit; and

FIG. 8 shows a schematic diagram of a medical x-ray device.

### DETAILED DESCRIPTION

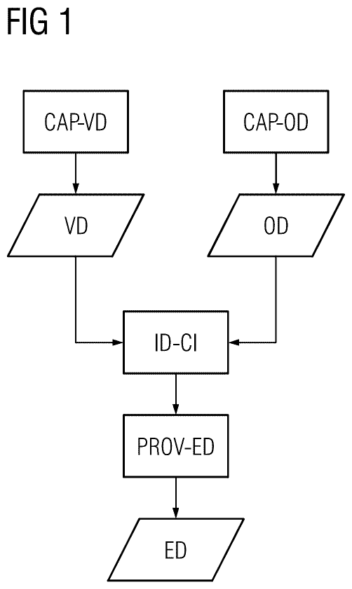

FIG. 1 shows a schematic diagram of a form of embodiment of a method for provision PROV-ED of a result dataset ED. In this case, a vessel dataset VD may be captured CAP-VD. The vessel dataset VD may have time-resolved images of at least one vessel section of an examination object in a number of physiological phases. An object dataset OD may further be captured CAP-OD. The object dataset OD may have an image of a medical object in the examination object in at least one matching physiological phase of the number of physiological phases. The medical object may include a diagnostic and/or surgical instrument and/or an implant. A corresponding image in the vessel dataset VD may further be identified in each case for the images of the object dataset OD with matching physiological phase ID-CI. Hereafter, the result dataset ED may be provided PROV-ED by at least partial overlaying and/or mixing of the images of the object dataset OD with the corresponding images of the vessel dataset VD.

In one embodiment, the object dataset OD may have time-resolved images of the medical object in the examination object in a number of physiological phases. Further, one corresponding image in the vessel dataset VD may be identified in each case for each of the images of the object dataset OD with matching physiological phase.

The physiological phases may include various phases of a breath movement and/or a heart movement of the examination object.

In one embodiment, the identification ID-CI of the respective one corresponding image in the vessel dataset VD for the images of the object dataset OD with matching physiological phase may be based on a further similarity metric that assesses a similarity between images of the vessel dataset VD and images of the object dataset OD.

In one embodiment, the provision PROV-ED of the result dataset ED may include a registration of the images of the object dataset OD with the corresponding images of the vessel dataset VD.

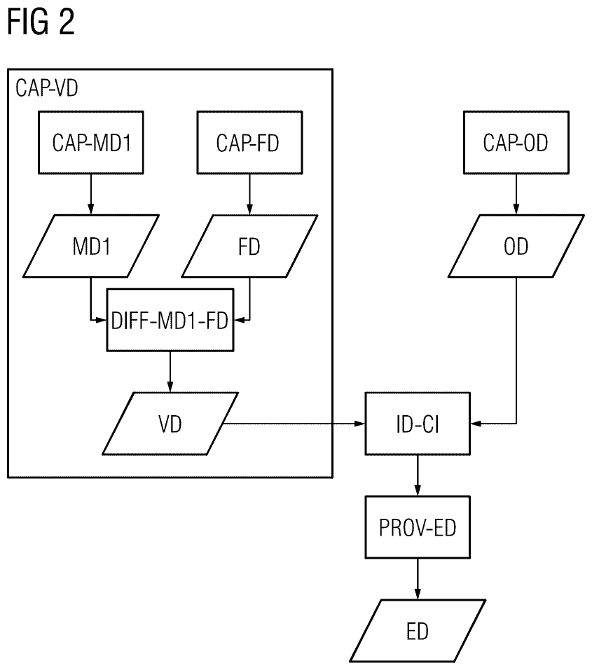

FIG. 2 shows a schematic diagram of a further form of embodiment of the method for provision PROV-ED of the result dataset ED. In this case, the capture CAP-VD of the vessel dataset VD may include a capture CAP-MD1 of a first mask dataset MD1 in a first mask phase and a capture CAP-FD of a fill dataset FD in a fill phase. In this case, the first mask dataset MD1 and the fill dataset FD may each have time-resolved images of a common examination region of the examination object in matching physiological phases. The common examination region may include the at least one vessel section. Further, in the fill phase, a contrast may be arranged in the at least one vessel section and depicted in the fill dataset FD. The vessel dataset VD may be provided as the difference DIFF-MD1-FD between images of the fill dataset FD and the first mask dataset MD1 with matching physiological phase in each case.

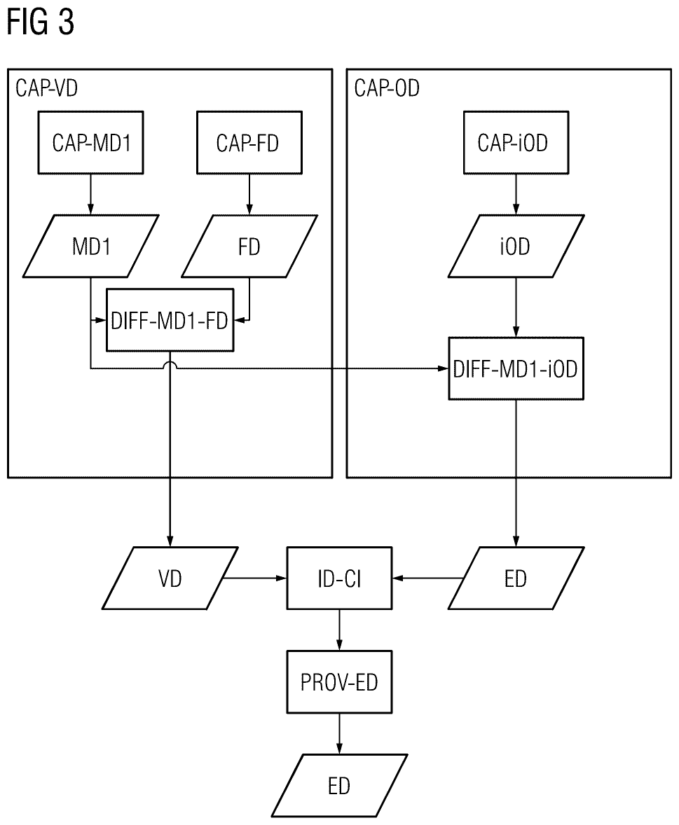

FIG. 3 shows a schematic diagram of a further form of embodiment of the method for provision PROV-ED of the result dataset ED. In this case, the capture CAP-OD of the object dataset OD may include a capture CAP-iOD of an initial object dataset in an object phase. In this case, the initial object dataset iOD may have an image of the common examination region of the examination object in the at least one matching physiological phase. Further, the medical object may be arranged in the object phase in the common examination region and be depicted in the initial object dataset. The object dataset OD may be provided as the difference DIFF-MD1-iOD between images of the initial object dataset iOD and the first mask dataset MD1 with matching physiological phase in each case.

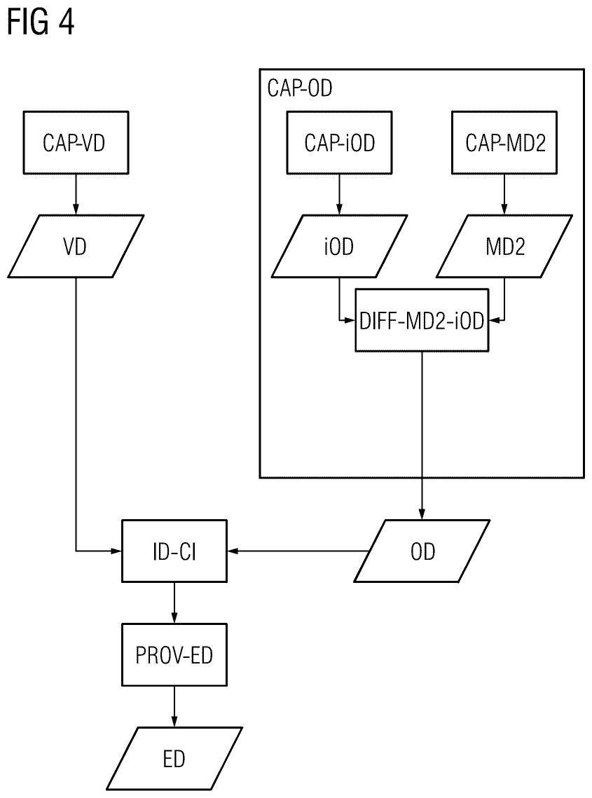

FIG. 4 shows a schematic diagram of a further form of embodiment of the method for provision PROV-ED of the result dataset ED. In this case, the capture CAP-OD of the object dataset OD may include a capture CAP-MD2 of a second mask dataset MD2 in a second mask phase and an initial object dataset iOD in an object phase. The second mask dataset MD2 and the initial object dataset iOD may each have an image of a common examination region of the examination object in at least one matching phase. The medical object may further be arranged in the object phase in the common examination region and be depicted in the initial object dataset. In this case, the object dataset OD may be provided as the difference between images of the initial object dataset iOD and the second mask dataset MD2 with matching physiological phase in each case.

In one embodiment, the images to be subtracted in each case may be identified with the respective matching physiological phase with the aid of a similarity metric, which assesses a similarity between images of various datasets.

The similarity metric and/or the further similarity metric may, for example, include a zero-normalized cross-correlation:

$$NC C ( {F}_{k} , {I}_{i} ) = {\sum }_{x , y} \frac{1}{{σ}_{{F}_{k}} {σ}_{{I}_{i}}} ( {F}_{k} ( x , y ) - {μ}_{{F}_{k}} ) ( {I}_{i} ( x , y ) - {μ}_{{I}_{i}} )$$

σ_{F}_{ₖ} and σ_{I}_{ᵢ} with the standard deviation and μ_{F}_{ₖ} and μ_{I}_{ᵢ} the averaged values over all pixel values Fₖ(x, y) and Iᵢ(x, y) of the corresponding images Fₖ and Iᵢ. In this case the imaging pair may be identified with a maximum value NCC as images to be subtracted with the respective matching physiological phase in each case.

Further, the respective images to be subtracted may be registered with one another.

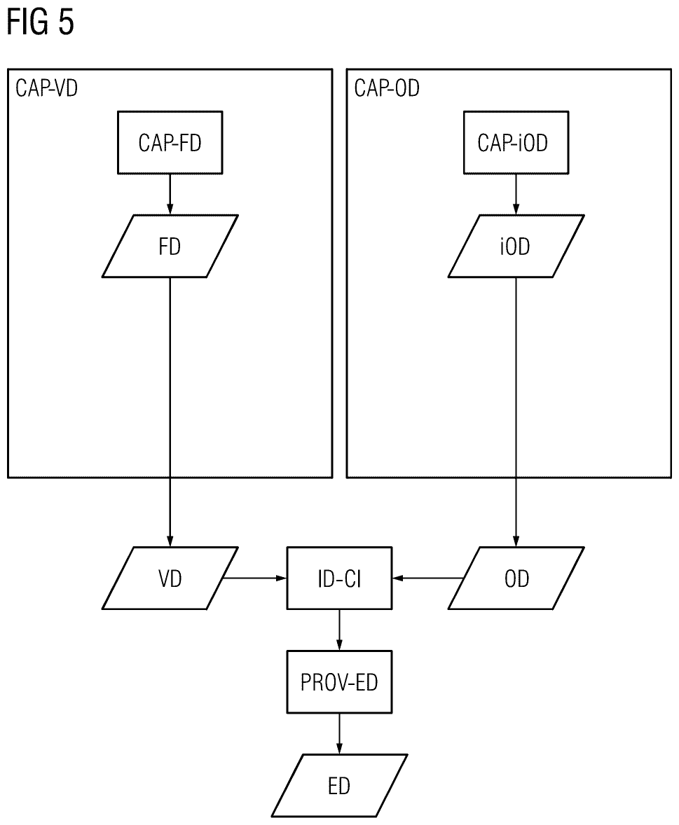

FIG. 5 shows a schematic diagram of a further form of embodiment of the method for provision PROV-ED of the result dataset ED. In this case, the capture CAP-VD of the vessel dataset VD may include a capture CAP-FD of a fill dataset FD in a fill phase. Further, the capture CAP-OD of the object dataset OD may include a capture CAP-iOD of an initial object dataset iOD in an object phase. The fill dataset FD and the initial object dataset iOD may each include an image of the common examination region in at least one matching physiological phase. The common examination region may include the at least one vessel section. A contrast may further be arranged in the at least one vessel section in the fill phase and be depicted in the fill dataset FD. The medical object may further be arranged in the object phase in the common examination region and be depicted in the initial object dataset iOD. The result dataset ED may be provided by at least partial overlaying and/or mixing of the images of the initial object dataset iOD with the corresponding images of the fill dataset FD.

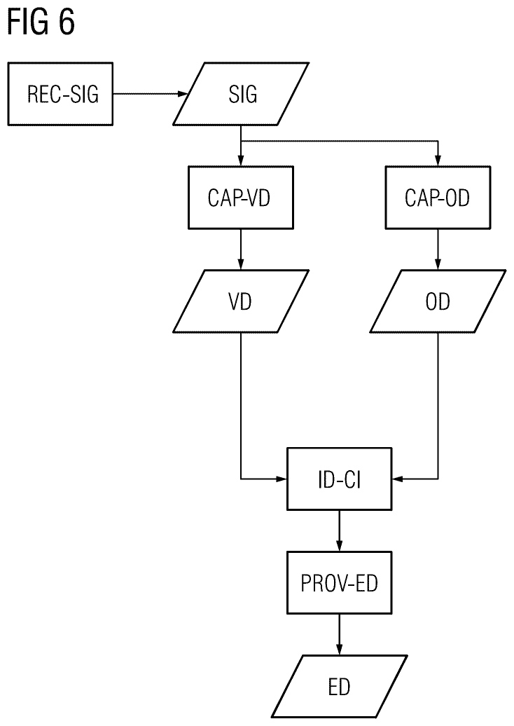

FIG. 6 shows a schematic diagram of a further form of embodiment of the method for provision PROV-ED of the result dataset ED. In this case, a physiological movement signal SIG, featuring information about a current physiological phase of the examination object, may be received REC-SIG. The vessel dataset VD and/or the object dataset OD may further be captured CAP-VD and CAP-OD depending on the physiological movement signal SIG.

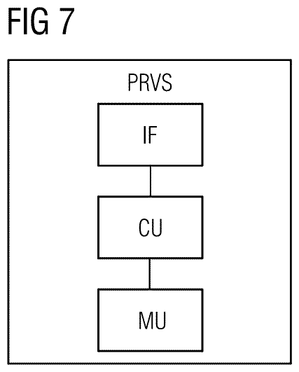

FIG. 7 shows a schematic diagram of one embodiment of a provision unit PRVS. In this case, the provision unit PRVS may include a computing unit CU, a memory unit MU, and/or an interface IF. The provision unit PRVS may be configured to carry out a method for providing a result dataset PROV-ED, in which the interface IF, the computing unit CU, and/or the memory unit MU are configured to carry out the corresponding method acts.

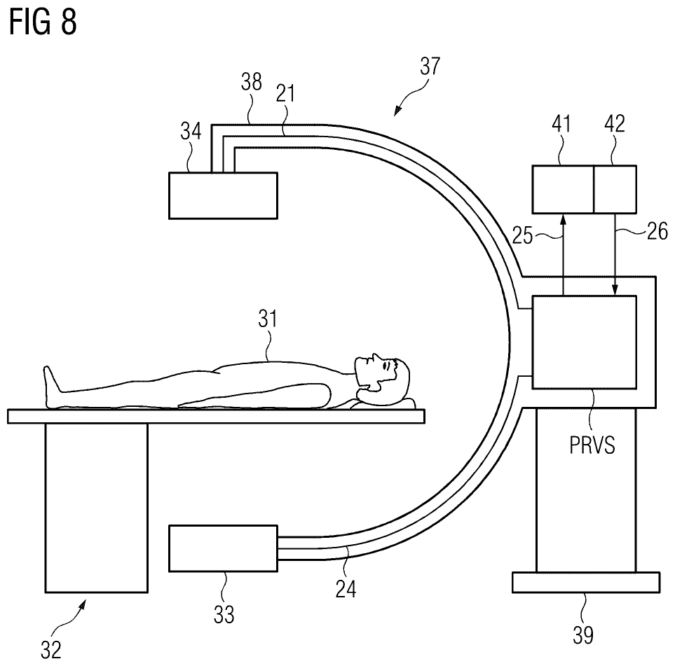

FIG. 8 shows, by way of an example of a medical imaging device, a schematic diagram of a medical C-arm x-ray device 37 including a provision unit PRVS of the present embodiments. The medical C-arm x-ray device 37 may have a detector 34 (e.g., an x-ray detector) and a source 33 (e.g., an x-ray source) that are arranged in a defined arrangement on a C-arm 38. The C-arm 38 of the C-arm x-ray device 37 may be supported for movement about one or a number of axes. For recording of the vessel dataset VD and of the object dataset OD of the examination object 31 positioned on a patient couch apparatus 32, the provision unit PRVS may send a signal 24 to the x-ray source 33. Thereafter, the x-ray source 33 may emit a bundle of x-rays. When the x-ray bundle, after interacting with the examination object 31, strikes a surface of the detector 34, the detector 34 may send a signal 21 to the provision unit PRVS. With the aid of the signal 21, the provision unit PRVS may capture, CAP-VD and CAP-OD, the vessel dataset VD and the object dataset OD.

The imaging device may further have an input unit 42 (e.g., a keyboard) and a visual display unit 41 (e.g., a monitor and/or a display and/or a projector). The input unit 42 may be integrated into the visual display unit 41 (e.g., with a capacitive and/or resistive input display). The input unit 42 may be configured for detecting a user input. For this, the input unit 42 may, for example, send a signal 26 to the provision unit PRVS. The provision unit PRVS may be configured to be controlled as a function of the user input (e.g., of the signal 26, such as for carrying out a method for providing a result dataset PROV-ED).

The visual display unit 41 may be configured to show a graphical display of the result dataset ED. For this, the provision unit PRVS may send a signal 25 to the visual display unit 41.

The schematic diagrams contained in the described figures do not depict scale or size relationships of any kind.

The method described in detail above, as well as the apparatus shown, merely represent exemplary embodiments that may be modified by the person skilled in the art in a very wide variety of ways, without departing from the area of the invention. Further, the use of the indefinite article “a” or “an” does not exclude the features concerned also being able to be present a number of times. Likewise, the terms “unit” and “element” do not exclude the components involved consisting of a number of interacting sub-components that may, if necessary, also be spatially distributed.

The expression “based on” may be understood in the context of the present application, such as in the sense of the expression “using”. For example, a formulation, according to which a first feature is created (e.g., alternatively, established, determined, etc.) based on a second feature, does not exclude that the first feature may be created (e.g., alternatively, established, determined, etc.) based on a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for providing a result dataset, the method comprising:
capturing a vessel dataset, wherein the vessel dataset has time-resolved images of at least one vessel section of an examination object in a number of physiological phases;
capturing an object dataset, wherein the object dataset has an image of a medical object in the examination object in at least one matching physiological phase of the number of physiological phases;
identifying in each case of a corresponding image in the vessel dataset for the images of the object dataset with matching physiological phase; and
providing the result dataset, the providing of the result dataset comprising at least partial overlaying, mixing, or overlaying and mixing of the images of the object dataset with the corresponding images of the vessel dataset.

2. The method of claim 1, wherein the object dataset has time-resolved images of the medical object in the examination object in a number of physiological phases,
wherein a corresponding image in the vessel dataset is identified in each case for each of the images of the object dataset with matching physiological phase.

3. The method of claim 1, wherein capturing the vessel dataset comprises capturing a first mask dataset in a first mask phase and of a fill dataset in a fill phase,
wherein the first mask dataset and the fill dataset each have time-resolved images of a common examination region of the examination object in matching physiological phases, wherein the common examination region comprises the at least one vessel section,
wherein, in the fill phase, a contrast is arranged in the at least one vessel section and is depicted in the fill dataset, and
wherein the vessel dataset is provided as a difference between images of the fill dataset and the first mask dataset with matching physiological phase in each case.

4. The method of claim 3, wherein capturing the object dataset comprises capturing an initial object dataset in an object phase,
wherein the initial object dataset has an image of the common examination region of the examination object in the at least one matching physiological phase,
wherein the medical object is arranged in the object phase in the common examination region and is depicted in the initial object dataset, and
wherein the object dataset is provided as a difference between images of the initial object dataset and the first mask dataset with matching physiological phase in each case.

5. The method of claim 3, wherein capturing the object dataset comprises capturing a second mask dataset in a second mask phase and an initial object dataset in an object phase,
wherein the second mask dataset and the initial object dataset each have an image of a common examination region of the examination object in at least one matching physiological phase,
wherein the medical object is arranged in the object phase in the common examination region and in the initial object dataset, and
wherein the object dataset is provided as a difference between images of the initial object dataset and of the second mask dataset with matching physiological phase in each case.

6. The method of claim 3, wherein the respective images to be subtracted are identified with the respective matching physiological phase with the aid of a similarity metric that assesses a similarity of images of various datasets.

7. The method of claim 3, wherein the respective images to be subtracted are registered with one another.

8. The method of claim 1, wherein capturing the vessel dataset comprises capturing a fill dataset in a fill phase,
wherein capturing the object dataset comprises capturing an initial object dataset in an object phase,
wherein the fill dataset and the initial object dataset each have an image of a common examination region of the examination object in at least one matching physiological phase,
wherein the common examination region comprises the at least one vessel section,
wherein, in the fill phase, a contrast is arranged in the at least one vessel section and is depicted in the fill dataset,
wherein the medical object is arranged in the object phase in the common examination region and is depicted in the initial object dataset, and
wherein the result dataset is provided by at least partial overlaying, mixing, or overlaying and mixing of the images of the initial object dataset with the corresponding images of the fill dataset.

9. The method of claim 1, wherein the physiological phases comprise various phases of a breath movement, heart movement, or breath movement and heart movement of the examination object.

10. The method of claim 1, wherein the medical object comprises a diagnostic, surgical, or diagnostic and surgical instrument, an implant, or a combination thereof.

11. The method of claim 1, wherein identifying the corresponding image in the vessel dataset in each case for the images of the object dataset with matching physiological phase is based on a further similarity metric that assesses a similarity of images of the vessel dataset with images of the object dataset.

12. The method of claim 1, wherein providing the result dataset comprises a registration of the images of the object dataset with the corresponding images of the vessel dataset.

13. The method of claim 1, further comprising receiving a physiological movement signal having information about a current physiological phase of the examination object,
wherein the vessel dataset, the object dataset, or the vessel dataset and the object dataset are captured depending on the physiological movement signal.

14. A provision unit for providing a result dataset, the provision unit comprising:
a processor configured to:
capture a vessel dataset, wherein the vessel dataset has time-resolved images of at least one vessel section of an examination object in a number of physiological phases;
capture an object dataset, wherein the object dataset has an image of a medical object in the examination object in at least one matching physiological phase of the number of physiological phases;
identify in each case of a corresponding image in the vessel dataset for the images of the object dataset with matching physiological phase; and
provide the result dataset, the processor being configured to provide the result dataset comprising the processor being configured to at least partially overlay, mix, or overlay and mix the images of the object dataset with the corresponding images of the vessel dataset.

15. A medical imaging device comprising:
a provision unit for providing a result dataset, the provision unit comprising:
a processor configured to:
capture a vessel dataset, wherein the vessel dataset has time-resolved images of at least one vessel section of an examination object in a number of physiological phases;
capture an object dataset, wherein the object dataset has an image of a medical object in the examination object in at least one matching physiological phase of the number of physiological phases;
identify in each case of a corresponding image in the vessel dataset for the images of the object dataset with matching physiological phase; and
provide the result dataset, the processor being configured to provide the result dataset comprising the processor being configured to at least partially overlay, mix, or overlay and mix the images of the object dataset with the corresponding images of the vessel dataset.

16. In a non-transitory computer-readable storage medium that stores instructions executable by one or more processors to provide a result dataset, the instructions comprising:
capturing a vessel dataset, wherein the vessel dataset has time-resolved images of at least one vessel section of an examination object in a number of physiological phases;
capturing an object dataset, wherein the object dataset has an image of a medical object in the examination object in at least one matching physiological phase of the number of physiological phases;
identifying in each case of a corresponding image in the vessel dataset for the images of the object dataset with matching physiological phase; and
providing the result dataset, the providing of the result dataset comprising at least partial overlaying, mixing, or overlaying and mixing of the images of the object dataset with the corresponding images of the vessel dataset.
